(19) United States
(12) Patent Application Publication
(10) Pub. No.: US 2006/0149203 A1
(43) Pub. Date: Jul. 6, 2006

(54) Disposable doggie diaper

(76) Inventors: Roland James Draper, Plano, TX (US); Margaret Ann Draper, Plano, TX (US)

Correspondence Address: Roland James and Margaret Ann Draper, 1401 Callaway Dr., Plano, TX, 75075-6806, US

(21) Appl. No.: 11/299,931

(22) Filed: Dec. 12, 2005

Related U.S. Application Data
(60) Provisional application No. 60/640,851, filed on Jan. 3, 2005.

(51) Int. Cl. A61F 13/15

(52) U.S. Cl. 604/385.09

(57) ABSTRACT

This invention, which is for a disposable doggie diaper, was created from our own need in the recent past. Our little dog had surgery near his rectal area, and the small amount of continued bleeding during post surgery was causing a problem, but in searching we found there was no disposable diaper on the market made specifically for dogs. We modified a baby disposable diaper to fit by cutting a hole in the rear and adjusting the size to fit our little Shih-Tzu dog. It worked like a charm. From that experience, we created this invention of the disposable doggie diaper for dogs to provide a simple solution for other dog owners. We have discussed this invention with other dog owners and veterinarians and received very positive results, They all agree there is a definite need for the disposable doggie diaper.

### CROSS-REFERENCE TO RELATED APPLICATION

U.S. Patent and Trademark Office provisional application No. 60/640,851 with filing date Jan. 3, 2005—Title: “Disposable Doggie Diaper Pants”

This provisional application pertains to this submission for a utility patent, and was granted to Roland James Draper and Margaret Ann Draper.

### STATEMENT REGARDING FEDERALLY SPONSORED RESEARCH

“Not Applicable”

### REFERENCE TO SEQUENCE LISTING, A TABLE, OR A COMPUTER PROGRAM LISTING COMPACT DISC APPENDIX

“Not Applicable”

### BACKGROUND OF THE INVENTION

This invention pertains to the pet care industry, and specifically to dogs. There has never been a satisfactory, efficient, and sanitary method to handle the problems described in the claims section of this filing. Problems can be extensive from toilet irregularities to post-surgery.

### BRIEF SUMMARY OF THE INVENTION

The disposable diaper for dogs is the solution to the problems previously stated above. The disposable diaper gives protection to those dogs (puppies and adult dogs) that have untrained toilet habits, females in season, incontinence, excitable urination, diarrhea, and blood or drainage from any sores or post-surgery in the area covered by the disposable diaper. The diaper is easy to put on the dog, and provides comfortable leak proof protection. The diaper is also easy to remove from the dog and it can be disposed of without creating a sanitary and unpleasant situation.

### BRIEF DESCRIPTION OF THE SEVERAL VIEWS OF THE DRAWING

FIG. 1-4 Open and flat view of the inside of the disposable diaper.

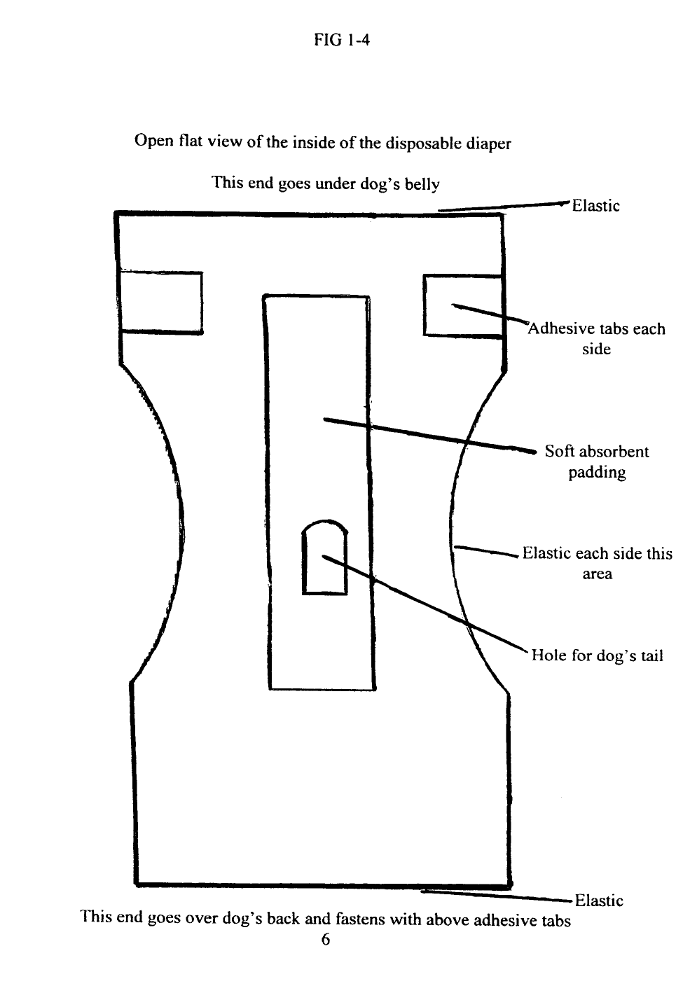

FIG. 2-4 A side view of the disposable doggie diaper on a dog. The diaper provides a secure snug fit.

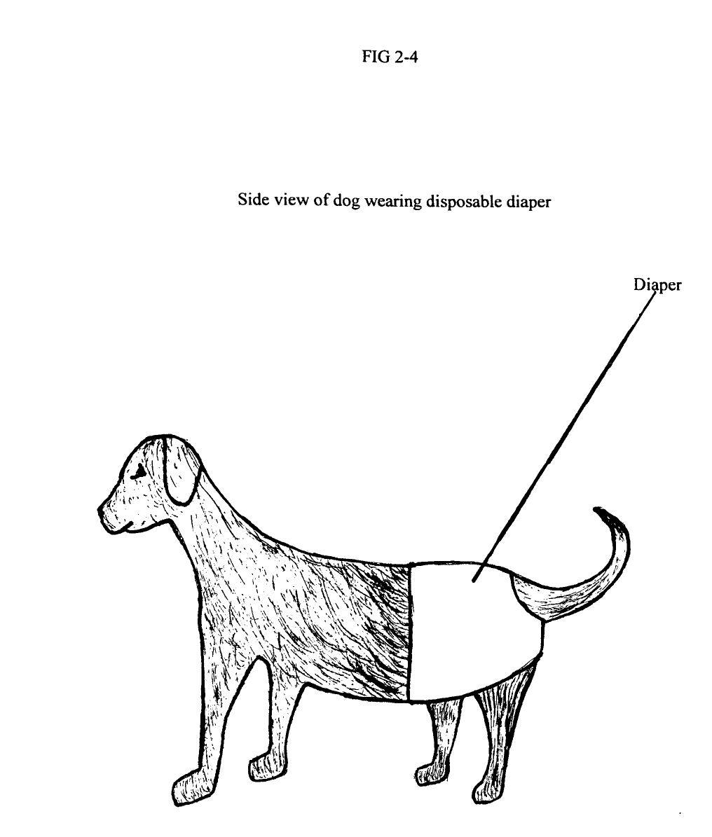

FIG. 3-4 This is a rear view of the dog wearing the disposable doggie diaper. It shows the opening (hole) in the rear which allows the tail to be outside the diaper.

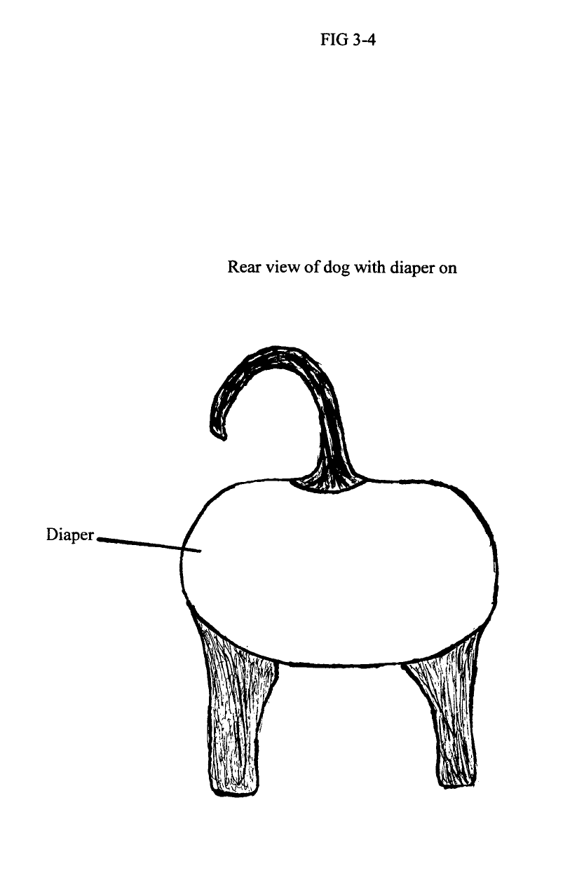

FIG. 4-4 Rear view of folded diaper with hole for tail.

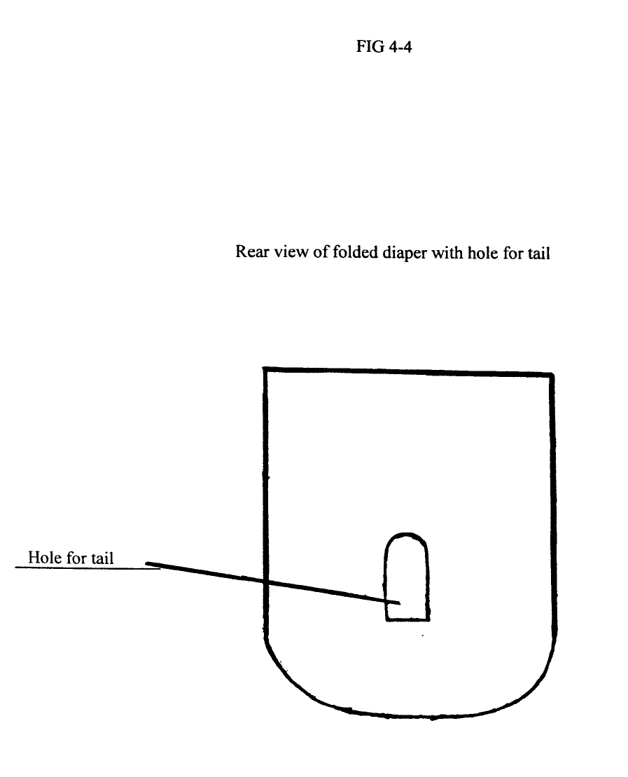

### DETAILED DESCRIPTION OF THE INVENTION

The disposable diaper for dogs is very similar to those diapers now used by humans. There would be an opening (hole) in the rear of the diaper to allow the dog's tail to be outside the diaper. The diaper can be put on the dog easily by slipping the tail of the dog through the hole in the diaper, and then placing the diaper between the back legs and firmly against the underside (belly) of the dog. Then, the adjustable adhesive tabs are brought up over the dog's back and secured to the top of the diaper. The diaper can easily be removed by pulling the adhesive tabs loose on both sides.

The diapers will be manufactured on the same type of equipment as used for disposable diapers for humans, except the diapers will be modified to fit a dog and provide an opening (hole) at the rear to accommodate the dog's tail. Composition of the material to produce the disposable diaper will be essentially of the same material presently used in human disposable diapers. The disposable diapers will be produced in different sizes based upon weight and waist (girth and loin) of the dog.

1. This claim is for a garment (disposable doggie diaper) which would be very valuable to dog owners and dog caretakers in that it prevents dogs from soiling property.
The disposable doggie diaper is the most effective and sanitary method for dogs who have problems with incontinence, menstruation, excitable urination, training problems, rectal bleeding, diarrhea, post surgery, or any bowel or urination problem.
There would be no messy clean-up required when using the disposable diaper, and the dirty diaper could be disposed of easily.
